(19) United States
(12) Patent Application Publication
(10) Pub. No.: US 2008/0269762 A1
(43) Pub. Date: Oct. 30, 2008

(54) METHOD AND DEVICE FOR REPAIR OF CARTILAGE DEFECTS

(75) Inventors: Bruce Simon, Mountain Lakes, NJ (US); Jennifer E. Woodell-May, Warsaw, IN (US)

Correspondence Address: HARNESS, DICKEY & PIERCE, P.L.C., P.O. BOX 828, BLOOMFIELD HILLS, MI, 48303, US

(73) Assignee: Biomet Manufacturing Corp., Warsaw, IN (US)

(21) Appl. No.: 11/740,014

(22) Filed: Apr. 25, 2007

(51) Int. Cl. A61B 17/88

(52) U.S. Cl. 606/99; 623/13.17; 623/14.12

(57) ABSTRACT

Methods for repairing a cartilage defect in a subject, such methods comprising placing a tissue specimen into a container, centrifuging the container to separate the specimen into at least three fractions, drawing a selected fraction from the container, processing the fraction into a therapeutic composition, and treating the cartilage defect with the therapeutic composition.

### INTRODUCTION

The present technology relates to methods, compositions, and devices for repairing cartilage defects.

Cartilage defects can appear as a hole or a tear in a cartilage surface and can result from wear, trauma or disease. Since cartilage has minimal ability to repair itself, even a small cartilage defect, if left untreated, can hinder a person's ability to move free from pain and can cause deterioration of a joint surface. Traditional treatments for cartilage defects include trimming the defect from the surface of the cartilage using orthoscopic surgery, or repairing the defect with sutures. More recently, treatments for cartilage defects have included the harvesting of cartilage cells, which are then cultured and implanted back into the cartilage defect to regenerate cartilage.

### SUMMARY

The present technology provides methods for repairing a cartilage defect in a human or animal subject. Such methods include a method for treating a cartilage defect comprising: obtaining blood compatible with the subject; fractionating the blood to produce platelet-poor plasma; concentrating the platelet-poor plasma to produce a platelet-poor plasma concentrate; and administering the concentrate to the site of the cartilage defect. The blood may be obtained from the subject and fractionated by centrifuging the blood to form platelet-poor plasma. The centrifuging may be performed using a container including a buoy that is able to separate the blood into two or more fractions having different densities.

Some methods further comprise administering to the cartilage defect an adjunct therapeutic material. The adjunct therapeutic material may be selected from the group consisting of bioactive agents, scaffold materials, isolated tissue materials, and combinations thereof. Bone marrow aspirate is used as a therapeutic material in some methods.

Further areas of applicability will become apparent from the description provided herein. It should be understood that the description and specific examples are intended for purposes of illustration only and are not intended to limit the scope of the present technology.

### DRAWINGS

The present technology will become more fully understood from the detailed description and the accompanying drawings, wherein:

FIG. 1 illustrates a representative site of a cartilage defect in a subject in need of treatment according to some embodiments of the present technology;

FIG. 2 is a diagrammatic illustration of a representative method for treating a cartilage defect according to one embodiment of the present technology;

FIG. 3 is a cross-sectional view of the representative device used for isolating a blood component according to one embodiment of the present technology;

FIGS. 4A and 4B are cross-sectional views of a representative device used for forming a therapeutic composition according to one embodiment of the present technology;

FIG. 5 illustrates a representative manner of administrating a cartilage defect treatment to the subject according to one embodiment of the present technology; and

FIG. 6 is a perspective view of a kit useful for treating a cartilage defect according to one embodiment of the present technology.

### DETAILED DESCRIPTION

The following description of technology is merely exemplary in nature of the subject matter, manufacture, and use of one or more inventions, and is not intended to limit the scope, application, or uses of any specific invention claimed in this application or in such other applications as may be filed claiming priority to this application, or patents issuing therefrom.

FIG. 1 is an example of a cartilage defect 130 in a human subject 100. The cartilage defect 130 is in a knee which comprises a femur 102, a tibia 103, a fibula 105, a patella 106, and cartilage 120. It should be understood, however, that the cartilage defect may be in any joint of a human subject 100 or animal subject, including shoulders, elbows, wrists, ankles, hips, and the spinal column, in which cartilage tissue is inadequate for physiological or cosmetic purposes. In this regard, cartilage defects include congenital cartilage defects, cartilage defects that result from or are symptomatic of disease, disorder, or trauma, and cartilage defects that are consequent to surgical or other medical procedures. For example, cartilage defects may be defects resulting from osteoporosis, spinal fixation procedures, hip and other joint replacement procedures, and chronic wounds.

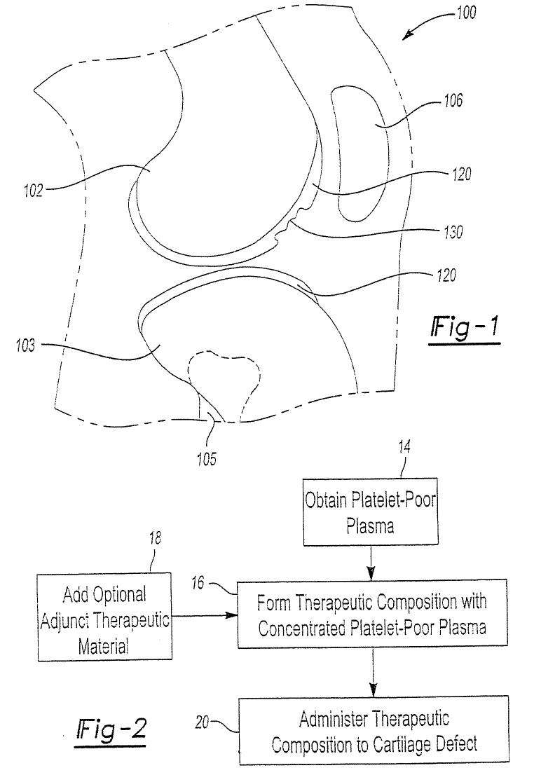

One embodiment for treatment of a cartilage defect 130 is shown diagrammatically in FIG. 2. In summary, platelet-poor plasma is obtained in step 14. The platelet-poor plasma is then processed in step 16 to form a therapeutic composition comprising concentrated platelet-poor plasma. An adjunct therapeutic material may also be combined with the concentrated platelet-poor plasma in step 18. The therapeutic composition from step 16 is then administered to a cartilage defect 130 in step 20, as further shown in FIG. 5. Each of the aforementioned steps will be more fully discussed below.

As discussed above, platelet-poor plasma is obtained at step 14. The platelet-poor plasma is preferably isolated from blood obtained from the subject 100 exhibiting the cartilage defect 130 to be treated. The blood may also be bone marrow derived. Platelet-poor plasma can be isolated in step 14 by a variety of methods, including by density fractionation of blood, cryopreciptation, and filtration. Density fractionation includes single stage centrifugation, centrifugation in multiple stages, and continuous flow centrifugation.

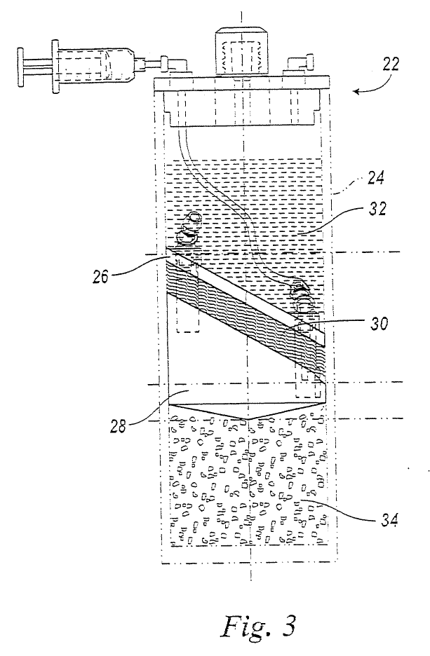

FIG. 3 illustrates one example of a separation device that can be used for forming the platelet-poor plasma in step 14 by density fractionation. In this regard, the device 22 includes a container 24, such as a tube, that is placed in a centrifuge after being filled with blood. The container 24 includes a buoy system having an isolator 26 and a buoy 28. The buoy 28 has a selected density which is tuned to reach a selected equilibrium position upon centrifugation; this position lies between a more dense blood fraction and a less dense blood fraction. During centrifugation, the buoy 28 separates the blood within the container 24 into at least two fractions, without substantially commingling the fractions, by sedimenting to a position between the two fractions. In this regard, the isolator 26 and the buoy 28 define a layer comprising platelet-rich plasma 30, while less dense platelet-poor plasma 32 generally fractionates above the isolator 26, and more dense red blood cells 34 generally fractionate below the buoy 28. Following centrifugation, a syringe or tube may then be interconnected with a portion of the buoy system to extract one or more selected fractions for use as the blood component. Devices including those disclosed in FIG. 3 and associated methods are described in U.S. Patent Application Publication 2004/0251217, Leach et al., published Dec. 12, 2004; and U.S. Patent Application Publication 2005/0109716, Leach et al., published May 26, 2005; both of which are incorporated by reference herein. One such device that is commercially available is the GPS® Platelet Concentrate System, from Biomet Biologics, Inc. (Warsaw, Ind.).

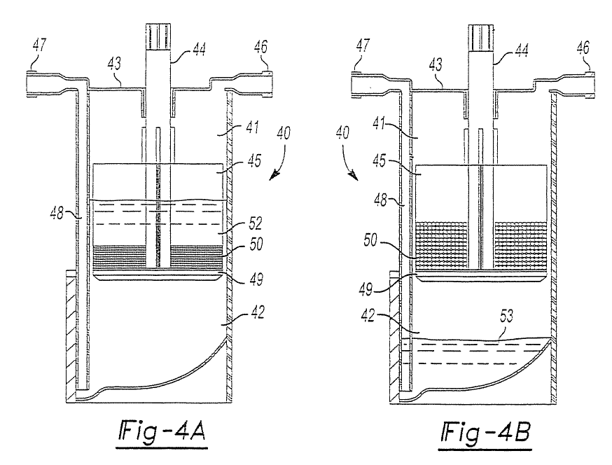

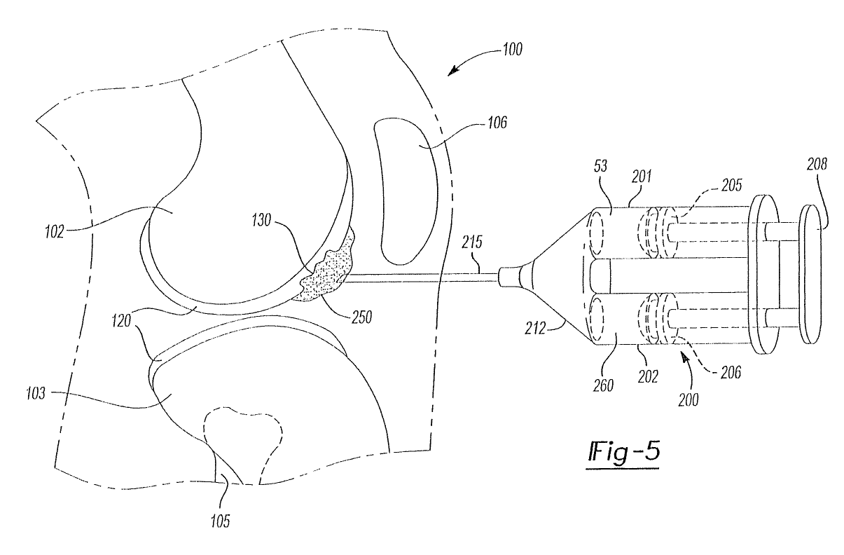

The platelet-poor plasma obtained in step 14 is processed into a therapeutic composition comprising concentrated platelet-poor plasma in step 16. One example of a concentration device 40 that may be used for forming concentrated platelet-poor plasma in step 16 is shown in FIGS. 4A and 4B. In this regard, the concentration device 40 has an upper chamber 41 and a lower chamber 42. The upper chamber 41 has an end wall 43 through which the agitator stem 44 of a gel bead agitator 45 extends. The concentration device 40 also has a plasma inlet port 46 that extends through the end wall 43 and into the upper chamber 41. The concentration device 40 also includes an outlet port 47 that communicates with a plasma concentrate conduit 48. The floor of upper chamber 41 includes a filter 49, the upper surface of which supports desiccating gel beads 50.

During use, platelet-poor plasma 52, with or without optional materials such as an adjunct therapeutic material discussed below, is introduced into the upper chamber 41 through the plasma inlet port 46. The platelet-poor plasma 52 flows to the bottom of the upper chamber 41 where it contacts the polyacrylate beads 50 as shown in FIG. 4A. As the polyacrylate beads 50 remove water from the platelet-poor plasma 52, the plasma 52 thickens. During this concentration stage, the platelet-poor plasma 52 and its components can be concentrated to a concentration of from about 1.5 to 3 times or higher than its original concentration to create the therapeutic composition 53.

Referring to FIG. 4B, the concentration device 40 is then placed in the cup receptors of a conventional laboratory centrifuge (not shown) and spun at a speed that will create a centrifugal force that will remove the therapeutic composition 53 from the polyacrylate gel beads 50, and cause the therapeutic composition 53 to flow through the filter 49. The filter 49 can be constructed to allow flow of liquid there-through at centrifugal forces above 10 g. After centrifugation is completed, the concentration device 40 is removed from the centrifuge. The platelet-poor plasma therapeutic composition 53 is then drawn from the lower chamber 42 through the conduit 48 to the outlet port 47. In some embodiments, the therapeutic composition 53 forms a gel.

Exemplary plasma concentration devices are disclosed in U.S. Patent Application Publication 2006/0175268, Dorian et al., published Aug. 10, 2006; and U.S. Patent Application Publication 2006/0243676, Swift et al., published Nov. 2, 2006; both of which are incorporated by reference herein. Such a device is commercially available as Plasmax™ Plus Plasma Concentrator, from Biomet Biologics, Inc. (Warsaw, Ind.).

The therapeutic composition obtained in step 16 may include optional materials that are combined with concentrated platelet-poor plasma in step 18. Optional materials include, for example, adjunct therapeutic materials such as platelet activators or other bioactive agents, scaffolds, buffers, isolated tissue materials and combinations thereof. Such adjunct therapeutic materials may be added to platelet-poor plasma prior to concentration of the platelet-poor plasma in step 16, or may be added to the therapeutic composition after concentration of the platelet-poor plasma in step 16.

Isolated tissue materials useful as optional materials in step 18 comprise tissue material that has been extracted from a human or other animal subject and which, in some embodiments, has been subjected to processing prior to mixing with concentrated platelet-poor plasma. Examples of isolated tissue material include platelet-rich plasma or other blood component, bone marrow aspirate, concentrated bone marrow aspirate, and processed lipoaspirate cells. The isolated tissue material may contain hematopoietic stem cells, stromal stem cells, mesenchymal stem cells, endothelial progenitor cells, red blood cells, white blood cells, fibroblasts, reticulocytes, adipose cells, thrombocytes, and endothelial cells. The isolated tissue material may be autologous tissue, i.e., tissue from the subject 100 having the cartilage defect 130 to be treated.

The isolated tissue material of step 18 may comprise bone marrow aspirate or concentrated bone marrow aspirate. Bone marrow aspirate can be obtained in any appropriate manner, such as from the intramedullary area of a bone by use of a syringe and needle. The bone marrow aspirate may be used as-is in step 18, or may be further processed to create bone marrow concentrate or other isolated tissue composition. In some embodiments, a separation device, such as shown in FIG. 3, may be used to obtain a concentrated bone marrow aspirate comprising nucleated cells, such as red and white blood cells, bone marrow stromal cells, and mesenchymal stem cells. For example, a mixture of whole blood and bone marrow aspirate may be added to the separation device 22 shown in FIG. 3, and a buffy coat fraction (platelet-rich plasma 30) obtained that contains at least a 4 times greater concentration of nucleated cells from bone marrow. Methods of obtaining an isolated tissue composition from bone marrow aspirate are disclosed in U.S. Patent Application Publication No. 2006/0278588 to Woodell-May published Dec. 14, 2006, which is incorporated by reference herein.

Other devices that may be used to obtain the isolated tissue composition at step 18 are described, for example, in U.S. Pat. No. 6,398,972, Blasetti et al., issued Jun. 4, 2002; U.S. Pat. No. 6,649,072, Brandt et al., issued Nov. 18, 2003; U.S. Pat. No. 6,790,371, Dolecek, issued Sep. 14, 2004; U.S. Pat. No. 7,011,852, Sukavaneshvar et al., issued Mar. 14, 2006; U.S. Patent Application Publication 2005/0196874, Dorian et al., published Sep. 8, 2005; and U.S. Patent Application Publication 2006/0175242, Dorian et al., published Aug. 10, 2006. In addition to the GPS® Platelet Concentrate System, a variety of other commercially available devices may be used to obtain the isolated tissue composition at step 18, including the Megellan™ Autologous Platelet Separator System, commercially available from Medtronic, Inc. (Minneapolis, Minn.); SmartPReP™, commercially available from Harvest Technologies Corporation (Plymouth, Mass.); DePuy (Warsaw, Ind.); the AutoloGel™ Process, commercially available from Cytomedix (Rockville, Md.), and the Genesis CS component concentrating system, available from EmCyte Corporation (Fort Myers, Fla.).

The isolated tissue composition of step 18 may comprise stem cells, such as bone marrow-derived stem cells and adipose-derived stromal cells. Adipose-derived stromal cells may be obtained from processing of lipid tissue by standard liposuction and lipaspiration methods known in the art. Adipose tissue may also be treated with digestive enzymes and with chelating agents that weaken the connections between neighboring cells, making it possible to disperse the tissue into a suspension of individual cells without appreciable cell breakage. Following disaggregation, the adipose stromal cells may be isolated from the suspension of cells and disaggregated tissue. A device such as the GPS® Platelet Concentrate System, may be used to isolate adipose stromal cells.

Platelet activators optionally included in step 18 may serve to activate one or more growth factors within platelets that optionally may be in the therapeutic composition. Activation of the platelets by the platelet activators can be performed just prior to administration of the therapeutic composition, concomitant with administration of the therapeutic composition, or following administration of the therapeutic composition to the cartilage defect in step 20. Platelet activators among those useful herein include thrombin, calcium chloride (CaCl₂), coagulation factors, and mixtures thereof. Coagulation factors include, but are not limited to, one or more of the following: V, VII, VIIa, IX, IXaβ, X, Xa, XI, XIa, XII, α-XIIa, β-XIIa, and XIII.

A scaffold may be added in step 18 to contain, support, or retain the therapeutic composition at the cartilage defect site, or to facilitate migration of endogenous cells into the administration site. Scaffolds may be formed from porous or semi-porous, natural, synthetic or semisynthetic materials. Scaffold materials include those selected from the group consisting of bone (including cortical and cancellous bone), demineralized bone, ceramics, polymers, and combinations thereof. Bone, demineralized bone and ceramics may be particularly useful in methods where the therapeutic composition is applied to subchondral bone, as in a microfracture procedure. Suitable polymers may include collagen, including lyophilized or skin-derived collagen as disclosed in U.S. patent application Ser. No. 11/259,216 which is incorporated by reference herein. Polymers may also include gelatin, hyaluronic acid, chitosan, polyglycolic acid, polylactic acid, polypropylenefumarate, polyethylene glycol, and copolymers or combinations thereof. Ceramics include any of a variety of ceramic materials known in the art for use for implanting in bone, such as calcium phosphate (including tricalcium phosphate, tetracalcium phosphate, hydroxyapatite, and mixtures thereof).

Referring again to FIG. 2, the therapeutic composition 53 created in step 16 is administered to the cartilage defect 130 in step 20 according to any medically appropriate procedure. For example, as noted above, a microfracture procedure may be performed at the site of the cartilage defect 130 prior to administering the therapeutic composition in step 20. In such a method, the subchondral bone adjacent to the cartilage defect is breached, and the concentrate is administered to the site of the breach.

As illustrated in FIG. 5, a dual syringe device 200 may be employed in a medically appropriate procedure. The dual syringe device 200 includes a first barrel 201 and a second barrel 202, both of which are connected to a mixing chamber 212. A first plunger 205 is inserted into the first barrel 201 and a second plunger 206 is inserted into the second barrel 202. The first plunger 205 and the second plunger 206 are connected by a member 208. The mixing chamber 212 connects to a cannula 215. In some embodiments, the dual syringe device 200 contains concentrated platelet-poor plasma 53 in the first barrel 201, and an adjunct therapeutic material 260, such as a platelet activator, in the second barrel 202. During step 20 of administering the therapeutic composition, member 208 is pushed toward the mixing chamber 212 such that the contents of both the first barrel 201 and the second barrel 202 are pushed into the mixing chamber 212. The therapeutic composition 250 in the mixing chamber 212 is pushed through the cannula 215 onto the cartilage defect 130. In some embodiments, depending on the adjunct therapeutic material 260, the therapeutic composition 250 can form a clot.

In some embodiments, the dual syringe device 200 is used to pierce soft tissue of the subject 100 to repair the cartilage defect 130. An incision may be made in the subject 100 to allow entry of the cannula 215 so that the dual syringe device 200 may enter an area of the cartilage defect 130.

The present technology also provides a cartilage repair system comprising a consumable component of a separation device, such as the separation device 22 illustrated in FIG. 3, and a concentration device 40 illustrated in FIGS. 4A and 4B. The cartilage repair system can also include a surgical process component operable to facilitate treatment of the cartilage defect 130 in the subject 100.

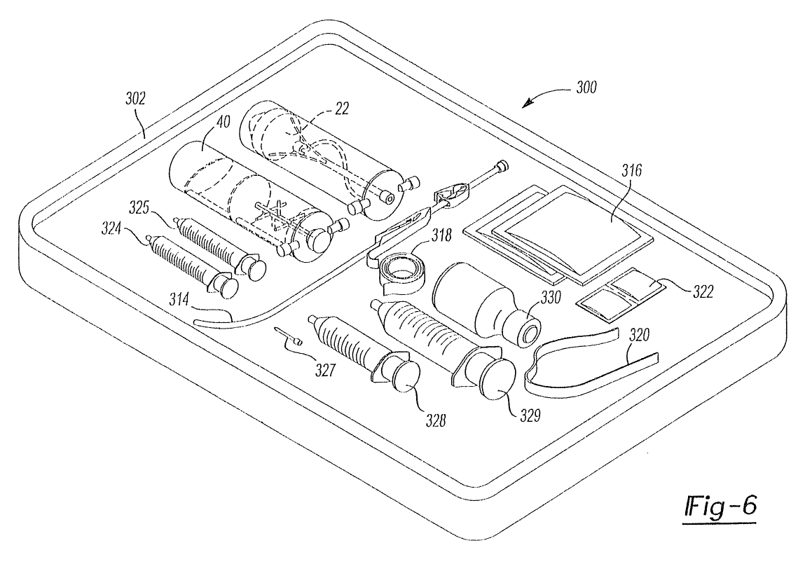

The present technology also provides kits to facilitate the methods described herein. As illustrated in FIG. 6, a kit 300 comprises one or more components, materials or devices used in such methods. A kit 300 can be placed in a tray 302 which is to provide a clean and sterile environment for use of the kit's contents during a method of the present technology.

The kit 300 may include a separation device 22 and a concentration device 40 such as illustrated in FIG. 3 and FIG. 4, respectively. The kit 300 also includes, for example, a first syringe 329 (e.g., with a 60 ml capacity) and a needle 327 to draw blood from the subject 100. The kit 300 may also include an anticoagulant solution 330, which may be drawn into first syringe 329 prior to drawing blood from the subject 100. The first syringe 329 can also be used to inject blood into the separation device 22. The kit may also contain a second syringe 328 (e.g., with a 30 ml capacity) for use in extracting platelet-poor plasma from the separation device 22 after centrifuging the separation device 22 with the blood. The platelet-poor plasma in the second syringe 328 may be injected into the concentration device 40. The kit may contain a third syringe 324 (e.g., with a 10 ml capacity) for use in withdrawing platelet-rich plasma from the separation device 22 if platelet-rich plasma is desired for use as an adjunct therapeutic material. The kit further contains a fourth syringe 325 (e.g., having a capacity of 10 ml) for extracting the therapeutic composition containing platelet-poor plasma from concentration device 40.

The kit 300 can further include an infusion cannula 314 that may be used for administering the therapeutic composition, as well as other materials and devices to facilitate the methods of the present technology. For example, a tourniquet 320, gauze 316, tape 318, antiseptic wipes 322, or other medical supplies may be provided to assist the practitioner. In some embodiments, the kit 300 can include a dual syringe device 200 such as illustrated in FIG. 5. The kit 300 can also include an adjunct therapeutic material such as a platelet activator, or an anticoagulant 330 as discussed above.

The systems and kits of the present technology may also include means of communicating information and/or instructions. The communication means may include language as required by an organization or government agency such as the United States Food & Drug Administration. The communication means can include labels; package inserts; brochures; advertisements; computer readable digital optical media, for example, diskettes or CD's; audio or video presentations, for example, audio tapes, CD's, or DVD's, and/or one or more pages on a website.

The embodiments and the examples described herein are exemplary and not intended to be limiting in describing the full scope of the devices, compositions and methods of the present technology. Equivalent changes, modifications and variations can be made within the scope of the present technology, with substantially similar results.

What is claimed is:

1. A method for treating a cartilage defect in a human subject comprising:
obtaining blood compatible with the subject;
fractionating said blood to produce platelet-poor plasma;
concentrating said platelet-poor plasma to produce a platelet-poor plasma concentrate; and
administering said concentrate to the site of said cartilage defect.

2. A method for treating a cartilage defect in a human subject according to claim 1, wherein said blood is obtained from said subject, and fractionating said blood to produce a blood component comprises centrifuging said blood to form said platelet-poor plasma.

3. A method for treating a cartilage defect in a human subject according to claim 2, wherein said centrifuging said blood to form said blood component comprises centrifuging blood in a container including a buoy that is able to separate said blood into two or more fractions having different densities.

4. A method for treating a cartilage defect in a human subject according to claim 1, further comprising administering to said cartilage defect an adjunct therapeutic material.

5. A method for treating a cartilage defect in a human subject according to claim 4, wherein said adjunct therapeutic material is selected from the group consisting of bioactive agents, scaffold materials, isolated tissue materials and combinations thereof.

6. A method for treating a cartilage defect in a human subject according to claim 5, wherein said adjunct therapeutic material is an autologous isolated tissue material selected from the group consisting of whole blood, platelet-rich plasma, lipoaspirate, adipose stromal cells, bone marrow aspirate, and combinations thereof.

7. A method for treating cartilage defect in a human subject according to claim 6, wherein said adjunct therapeutic material comprises concentrated bone marrow aspirate.

8. A method for treating a cartilage defect in a human subject according to claim 7, further comprising obtaining bone marrow from said subject, and centrifuging said bone marrow aspirate to produce said concentrated bone marrow aspirate.

9. A method for treating a cartilage defect in a human subject according to claim 5, wherein said adjunct therapeutic material comprises a platelet activator.

10. A method for treating a cartilage defect in a human subject according to claim 5, wherein said adjunct therapeutic material is a scaffold selected from the group consisting of collagen, hyaluronic acid, chitosan, demineralized bone matrix, bone graft materials, ceramics, and mixtures thereof.

11. A method for treating a cartilage defect in a human subject according to claim 1, further comprising performing a microfracture procedure at the site of said defect prior to said administering of said concentrate.

12. A method for treating a cartilage defect in a human subject according to claim 11, wherein said microfracture procedure breaches suchondral bone adjacent to said cartilage defect, and said concentrate is administered to the site of said breach.

13. A method for treating a cartilage defect in a human subject according to claim 12, wherein said concentrate further comprises a scaffold material selected from the group of demineralized bone matrix, bone graft materials, and ceramics.

14. A method for treating a cartilage defect in a human subject comprising:
obtaining blood from said subject;
centrifuging said blood to obtain platelet-poor plasma;
forming a platelet-poor plasma concentrate by desiccating and centrifuging said platelet-poor plasma;
combining said platelet-poor plasma gel with an adjunct therapeutic material to form a therapeutic composition; and
administering said therapeutic composition and a platelet activator to the site of said tissue defect.

15. The method for treating a cartilage defect in a human subject according to claim 14, wherein said centrifuging said blood to form said blood component comprises centrifuging blood in a container including a buoy that is able to separate said blood into two or more fractions having different densities.

16. The method for treating a cartilage defect in a human subject according to claim 14, wherein said adjunct therapeutic material is selected from the group consisting of bioactive agents, scaffold materials, isolated tissue materials and combinations thereof.

17. A cartilage repair system, comprising:
a blood separation device comprising a container having at least one buoy, said container and buoy being operable during centrifugation of said container to produce isolated platelet-poor plasma from said whole blood; and
a plasma concentration device operable to produce a platelet-poor plasma gel from said isolated platelet-poor plasma;
a surgical process component operable to facilitate the repair of a cartilage defect in a human or other animal subject using said gel.

18. A cartilage repair system according to claim 17, wherein said surgical process component is selected from the group consisting of application devices, adjunct therapeutic materials, and combinations thereof.

19. A cartilage repair system according to claim 18, wherein said system comprises an adjunct therapeutic material comprising a platelet activator.

20. A cartilage repair system according to claim 19, wherein said system comprises an application device comprising a syringe operable to co-administer said platelet-poor plasma gel and a platelet activator.

21. A kit for cartilage repair, comprising:
a plasma concentration device operable to produce a platelet-poor plasma concentrate; and
a blood separation device comprising a container having at least one buoy, said container and buoy being operable during centrifugation of said container to isolate platelet-poor plasma from said whole blood.

22. A kit for cartilage repair according to claim 21, further comprising a surgical process component selected from the group consisting of application devices, adjunct therapeutic materials, anticoagulant, and combinations thereof.

23. A kit for cartilage repair according to claim 22, wherein said kit comprises an adjunct therapeutic material.

24. A kit for cartilage repair according to claim 22, wherein said kit comprises an application device comprising a syringe operable to co-administer said platelet-poor plasma concentrate and a platelet activator.
